(19) United States
(12) Patent Application Publication
(10) Pub. No.: US 2009/0137031 A1
(43) Pub. Date: May 28, 2009

(54) Culturing Apparatus and Culturing Method for Photosynthesis Microorganism

(76) Inventor: Seishiro Hirabayashi, Shizuoka (JP)

Correspondence Address: STAAS & HALSEY LLP, SUITE 700, 1201 NEW YORK AVENUE, N.W., WASHINGTON, DC, 20005, US

(21) Appl. No.: 11/884,116

(22) PCT Filed: Feb. 10, 2005
(86) PCT No.: PCT/JP2005/002030

§ 371 Date: Aug. 10, 2007

(30) Foreign Application Priority Data
Sep. 29, 1999 (JP) 11/2777611

(51) Int. Cl. C12M 1/00; C12M 1/09; A01G 7/00

(52) U.S. Cl. 435/292.1; 435/293.1; 435/296.1; 47/1.4

(57) ABSTRACT

A culturing apparatus for photosynthesis microorganism provided by the present invention comprises a outer vessel, inner vessel, culture solution circulating portion and a heat transfer medium feeding portion. Culture solution containing photosynthesis microorganism is put into the outer vessel which is transparent and configured like a cylinder extending in a predetermined axis-direction. The inner vessel is disposed in the outer vessel, being configured like a cylinder extending in the above axis-direction. The culture solution circulating portion draws culture solution from one side along the axis-direction in the outer vessel, supplying that to the other side of the axis-direction. The heat transfer medium feeding portion supplies heat transfer medium into the inner vessel.

### FIELD OF INVENTION

The present invention relates to a culturing apparatus and a culturing method for photosynthesis microorganism.

### BACKGROUND

According to prior art methods, photosynthesis microorganism, for instance, algae such as Chlorella, Spirulina or Donarinila is cultured in culture solution to produce useful substances such as protein, polysaccharides, fatty acids or pigments, which are generated by such photosynthesis microorganism through photosynthesis.

Patent Documents 1 and 2 disclose apparatuses for applying methods of culturing photosynthesis microorganism efficiently. These apparatuses comprise vessels each of which is composed of a pair of transparent dome-like members piled up vertically, between which culture solution is stored as a thin layer. Then cooling water is sprinkled from above the upper-side transparent member to control temperature of the culture solution.

Patent Document 1; WO-99/50384 (International-Laid-Open Pamphlet)

Patent Document 2; WO01/023519 (International-Laid-Open Pamphlet)

### DISCLOSURE OF INVENTION

### Problem to be Solved by Invention

According to researches by the present inventor, however, the above-described apparatuses fail to culture photosynthesis microorganism efficiently because temperature control of photosynthesis microorganism is not enough.

The present invention has been proposed under such problem to be solved, aiming to provide a culturing apparatus and a culturing method which are capable of culturing photosynthesis microorganism efficiently under a high temperature controllability.

### Means for Solving Problem

A culturing apparatus for photosynthesis microorganism in accordance with the present invention comprises an outer vessel, inner vessel, a culture solution circulating portion and a heat transfer medium feeding portion.

The outer vessel is transparent and configured like a cylinder extending in a predetermined axis-direction, accommodating photosynthesis microorganism therein.

The inner vessel is disposed within the outer vessel and configured like a cylinder extending in the said axis-direction.

The culture solution circulating portion draws culture solution from one side along said axis-direction in the outer vessel and supplies the drawn culture solution to the other side along said axis-direction in the outer vessel.

The heat transfer medium feeding portion supplies heat transfer medium into the inner vessel.

A culturing method in accordance with the present invention is a method of culturing by the use of the above culturing apparatus for photosynthesis microorganism.

The present invention enables culture solution to be irradiated by light such as sun light from the outside of the transparent outer vessel efficiently since the culture solution containing photosynthesis microorganism flows in the axis-direction from one side to the other side through a gap between the outer vessel and the inner vessel as to form a relatively thin layer.

In addition, it is made easy to keep the temperature of the culture solution at a desirable temperature and maintain the temperature of the culture as to be suitable for culturing regardless of changing of circumstance because heat exchange is performed between the heat transfer medium fed into the inner vessel and the culture solution in the gap between the inner vessel and the outer vessel. Therefore, an efficient culturing of photosynthesis microorganism is realized.

Said heat transfer medium may be heating medium or cooling medium, allowing the culture solution to be heated or cooled.

Preferably, in the above-described invention, the outer vessel and the inner vessel are arranged coaxially. Further, both the outer vessel and the inner vessel are preferably cylindrical. This would make easy to form a tubular gap provided with a uniform thickness around the axis-direction between the outer vessel and the inner vessel, with the result that a heightened solution flow would uniformity is obtained and problems such as flow stopping or deposition onto a wall surface hardly arise.

It is further preferable that the inner vessel is made of a metal material or a glass material. The metal material or the glass material has a higher thermal conductivity as compared with those of resin materials or the like, which enables heat exchange between heat transfer medium and culture solution to be performed quickly, thereby ensuring much reliable temperature control of culture solution. On the other hand, an inner vessel made of a resin material gives a preferable situation that the inner vessel can be manufactured at a low cost.

The culture solution circulating portion is preferably provided with a transparent tube which is transparent and cylindrical and allows culture solution to flow therethrough. This would causing culture solution to have photosynthesis within the transparent tube, leading to a more efficient culturing.

Further, said axis-direction is preferably a vertical direction, that is, the outer vessel and the inner vessel extend preferably in a vertical direction. This could make the apparatus shaped oblong, thereby enabling a large quantity of culture solution to be processed under a small occupation area.

If the foresaid axis-direction is a vertical direction, the culture solution circulating portion is preferably provided with a transparent tube shaped like a cylinder and a first nozzle disposed at a lower part of the transparent tube, wherein the transparent tube extends in a vertical direction and allows culture solution to flow therethrough, and the first nozzle supplies a gas into the transparent tube.

If structured such, not only culture solution can have photosynthesis within the transparent tube too, resulting in an efficient culturing, but also gas supply from the first nozzle to the vertical transparent tube causes culture solution to be transferred due to an air-lift effect brought by ascending bubbles of gas supplied from the first nozzle, thereby enabling culture solution to be circulated between the outer vessel and the culture solution circulating portion without being equipped with a centrifugal pump or the like.

Therefore cells of photosynthesis microorganism can be prevented from being damaged. In particular, it is preferable that a gas such as air to which carbon dioxide is added is adopted as the above gas because carbon dioxide concentration in culture solution can be maintained at the same time.

In addition, if the axis-direction is a vertical direction, a second nozzle supplying a gas to a bottom part of the outer vessel may be disposed.

If a gas containing carbon dioxide is supplied from such a second nozzle at a degree such that circulation current between the outer vessel and culture solution circulating portion is not affected, a sufficient concentration of dissolved carbon dioxide can be maintained and culturing is achieved still more efficiently.

### ADVANTAGE OF INVENTION

The present invention provides a culturing apparatus s and a culturing method which is capable of culturing photosynthesis microorganism efficiently.

### BRIEF DESCRIPTION OF DRAWINGS

FIG. 1 is an outlined partial cross section view of a culturing apparatus for photosynthesis microorganism in accordance with the present invention;

FIG. 2 is a view taken in the direction of the arrows II-II in FIG. 1;

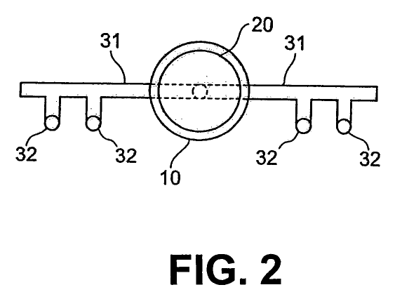

FIG. 3 is a diagram showing an example of relation between photosynthesis velocity of microorganism and light intensity;

FIG. 4 is a diagram showing an example of relation between thickness of culture solution and light attenuation;

FIG. 5 is a table showing a composition of culture solution employed in Embodiment 1;

FIG. 6 is a table showing a composition of culture solution employed in Embodiment 2;

FIG. 7 is a table showing a composition of culture solution employed in Embodiment 3;

FIG. 8 is a table showing a composition of culture solution employed in Embodiment 4;

FIG. 9 is a table showing a composition of culture solution employed in Embodiment 5;

FIG. 10 is a table showing a composition of culture solution employed in Embodiment 6; and

FIG. 11 is a table showing a composition of culture solution employed in Embodiment 7.

### REFERENCE SYMBOLS

C . . . culture solution
H . . . heat transfer medium
10 . . . outer[vessel
17 . . . second nozzle
20 . . . inner vessel
30 . . . culture solution circulating portion
32 . . . transparent tube
35 . . . first nozzle
40 . . . heat transfer medium feeding portion
100 . . . culturing apparatus for photosynthesis microorganism

### BEST MODE EMBODIMENTS OF INVENTION

Described hereafter in detail are preferable embodiments In addition, the present invention, with the drawings being referred as required. It is noted that the same elements are denoted by the same symbols in the drawings and repeated explanations are omitted. Illustration ratio shown in the drawings gives no limitation.

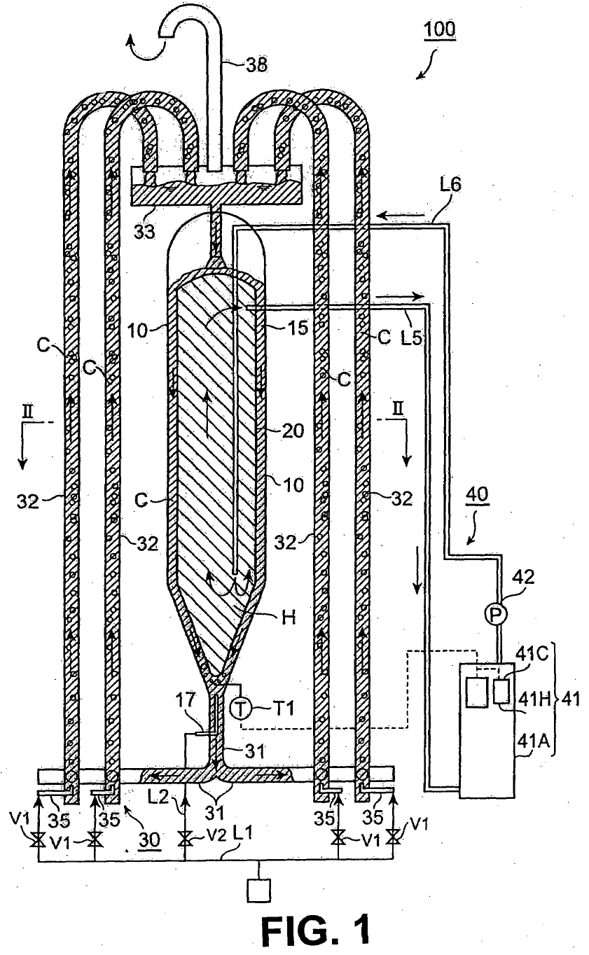

FIG. 1 is an outlined diagramic side view of a basic construction of a culturing apparatus in accordance with an embodiment of the present invention.

Culturing apparatus 100 for photosynthesis microorganism In addition, the present invention is an enclosure-type culturing apparatus main components of which are outer vessel 10, inner vessel 20 disposed within outer vessel 10, culture solution circulating portion 30 for circulating culture solution and heat transfer medium feeding portion 40.

Outer vessel 10 is a hollow container shaped like a cylinder extending in a vertical direction. A bottom portion thereof is closed by being configured like a cone tapering downward while an upper end portion is closed by being configured like a dome. Second nozzle 17 for feeding a gas containing carbon dioxide into outer vessel 10 is connected to the bottom portion of outer vessel 10.

Outer vessel 10, inner vessel 20 is made of a transparent material, allowing visible light of sun of ambient sunlight or the like, incident thereto, to transmit within outer vessel 10. The transparent material may be any material so far as it has a good transmissivity for visible light, a high weatherability and a ultraviolet ray resistance, being preferably, for example, resin such as acrylic resin, polycarbonate, polypropylene, polyethylene or polyvinyl chloride, or glass or the like.

Inner vessel 20 is accommodated in outer vessel 10, being configured like a cylinder extending coaxially. with outer vessel 10. An upper end portion of inner vessel 20 is closed by being configured like a dome while a lower end portion of inner vessel 20 is closed by being configured like a cone tapering downward as to correspond to an lower end portion of outer vessel 10. Outer vessel 10 is not in communication with inner vessel 20.

Although particular limitation is not applied to the material which inner vessel 20 is made of, metal materials having a high thermal conductivity or glass material is employed preferably. Examples of such metal materials are aluminum and stainless steel. Alternatively, if inner vessel 20 is made of any resin material as previously described, inner vessel 20 can be manufactured at a low cost. Cooling medium or heating medium is supplied into inner vessel 20 as heat transfer medium.

Tube-like gap 15 is formed as to extend in a vertical direction between an outer periphery surface of inner vessel 20 and an inner periphery surface of outer vessel 10. This gap 15 accommodates culture solution C containing photosynthesis microorganism such as algae. Thickness (radial distance) of gap 15 falls preferably in a range from 2 cm to 10 cm approximately. Thickness of gap 15 can be changed freely by changing the outer diameter of inner vessel 20. Thickness of gap 15 may be suitably chosen depending on kinds of photosynthesis microorganism to be cultured, concentration of photosynthesis microorganism in culture solution (culture concentration) or others. In addition, an example of employable range of diameter of outer vessel 10 is from 30 cm to 100 cm approximately.

Further, ring-like gap 15 may extend in a up-down direction by 2 cm to 10 cm approximately.

Still further, a photocatalyst layer is preferably coating—applied at least to one of an inner periphery surface of outer vessel 10 and an outer periphery surface of inner vessel 20.

The photocatalyst may be any material so far as it Although particular limitation is not applied to such photocatalyst so far as a photocatalyst reaction arises as to make the surface(s) hydrophilic, solid oxide semiconductors such as titan dioxide (TiO₂) or zinc oxide (ZnO) are employed preferably and, in particular, titan dioxide (TiO₂) is desirable. This enables photosynthesis microorganism to be restrained from stick to the surface(s) of outer vessel 10 and/or inner vessel 20.

Culture solution circulating portion 30 draws culture solution C in outer vessel 10 through a lower portion of outer vessel 10 and returns the drawn culture solution C to an upper portion of outer vessel 10, thereby functioning as a culture solution circulating means.

This culture solution circulating portion 30 comprises lower side manifold 31 connected to a lower end of outer vessel 10, a plurality of transparent tubes 32 connected to lower side manifold 31, upper side receiving vessel 33 connected not only to transparent tubes 32 and but also to an upper end of outer vessel 10 and first nozzles 35 disposed at respective lower portions of transparent tubes 32.

Lower side manifold 31 is a branching tube a tube-gathering end of which is connected to the lower end of outer vessel 10, and branched ends of which are connected to the lower end portions of transparent tubes 32, respectively. Lower side manifold 31 delivers culture solution C in outer vessel 10, after diverging into a plurality of parts, to the respective transparent tubes 32.

Transparent tubes 32 are transparent pipes extending vertically. The upper ends of transparent tubes 32 are bent downward like a letter U after extending as to be taller than outer vessel 10, having distal ends each of which is connected to upper side receiving vessel 33. Transparent tubes 32 are made of generally the same material as that of outer vessel 10. Diameter of each transparent tube 32 is smaller than that of outer vessel 10, which may fall in a range from 3 cm to 7 cm approximately.

First nozzles 35 are disposed at the lower end portions of transparent tubes 32. Line L1 is connected to first nozzles 35, which is connected to gas source 70 via valves V1. Gas source 70 feeds a gas containing carbon dioxide, such as air to which carbon dioxide is added. The gas being supplied from gas source 70 into transparent tubes 32 via first nozzles 35, the gas becomes bubbles which rise in transparent tubes 32, thereby transferring culture solution C upward through air lift effect, with the result that upper side receiving vessel 33 is supplied with culture solution C. It is noted that this gas source 70 is also connected to second nozzle 17 via line L2 provided with valve V2.

Upper side receiving vessel 33 is disposed above outer vessel 10, being a vessel receiving culture solution C sent from transparent tubes 32. A lower portion of upper side receiving vessel 33 is communicated with an upper part within outer vessel 10, and culture solution C received by upper side receiving vessel 33 is supplied into outer vessel 10, concretely, toward an upper end of inner vessel 20.

This upper side receiving vessel 33 is further provided with ventilation opening 38 which extends upward and has a distal end bent like a U-shape as to enable oxygen gas and others generated by culture solution C to be discharged. It is noted that culturing apparatus 100 is closed tightly except for this ventilation opening 38, thereby enabling contamination by other microorganism or dust to be restrained.

Heat transfer medium feeding portion 40 supplies heat transfer medium functioning as heating medium or cooling medium into inner vessel 20. This heat transfer medium feeding portion 40 comprises line L5, line L6, temperature controller 41 for regulating temperature of heat transfer medium H at a predetermined temperature and pump 42.

Temperature controller 41 comprises vessel 41A, heater 41H and chiller 41C, being capable of heating or chilling heat transfer medium H flowing through vessel 41A. More concretely, outer vessel 10 is provided with thermometer T1 for measuring temperature of culture solution C in outer vessel 10, and heater 41H or chiller 41C of temperature controller 41 controls temperature of heat transfer medium C so that temperature of culture solution C in outer vessel 10 is maintained within a predetermined range on the basis of data provided by thermometer T1.

An end of line L5 is connected to an upper portion of inner vessel 20 and the other end of line L5 is connected to vessel 41 of temperature controller 41. An end of line L6 is connected to a lower portion of inner vessel 20 and the other end of line L6 is connected to vessel 41 of temperature controller 41 via pump 42.

Pump 42 is arranged on line L6, circulating heating medium or cooling medium between inner vessel 20 and temperature controller 41.

The lower portion of inner vessel 20 is supplied, via pump 42 and line L6, with heat transfer medium temperature of which is regulated at a predetermined temperature by heater 41H or chiller 41C, the heat transfer medium then making an upward move within inner vessel 20 and causing heat exchange with culture solution C through a wall of inner vessel 20 to occur, with the result temperature of culture solution C is maintained within a predetermined range. Further to this, heat transfer medium H is discharged from the upper portion of inner vessel 20 to be returned to vessel 41A of temperature controller 41 via line L5, being heated or chilled again.

Particular limitation is not applied to heat transfer medium H and water, oil steam or the like may be employed.

Since heat transfer medium sent from temperature controller 41 flows upward from downside within inner vessel 20, heat transfer medium comes to contact with culture solution flowing downward from upside in a gap between inner vessel 20 and outer vessel 10 as to an against flow, thereby providing a high heat exchange ability.

Next, described is a method of culturing photosynthesis microorganism by the use of culturing apparatus 100.

In advance, culture solution C containing photosynthesis microorganism is poured into outer vessel 10 and culture solution circulating portion 30. Pouring quantity of culture solution C is generally enough if it causes air lift effect to be performed by a gas injected from first nozzles 35 within transparent tubes 32 and culture solution C is conveyed as to be circulated between culture solution circulating portion 30 and outer vessel 10. It is noted that culturing apparatus 100 is installed outdoors.

Although particular limitation is not applied to photosynthesis microorganism so far as performing photosynthesis is realized, especially preferable examples are algae, for instance, haptoalgae such as Isochrysis, green algae such as Haematococcus, Nannnochloropsis, Parietochloris or Chlorella, blue algae such as Spirulina, Nostoc, or brown algae such as Donarinila or Phaeodactylum.

Constituents contained in the culture solution other than photosynthesis microorganism may be suitable ones depending on kinds of photosynthesis microorganism, such as salts, vitamins.

In the next place, injected is a gas containing carbon dioxide, for instance, in a range from about 0.5 to 3.0 vol/vol %, such as air containing carbon dioxide, for instance, in a range from about 0.5 to 3.0 vol/vol %. to transparent tubes 32 from first nozzles 35.

This causes bubbles of the gas to ascend within transparent tubes 32, thereby moving culture solution C in transparent tubes 32 from downside to upside through air-lift effect, with the result that the upper portion of outer vessel 10 is supplied with culture solution C via upper side receiving vessel 33. Then culture solution C flows downward in gap 15 between outer vessel 10 and inner vessel 20, returning to transparent tubes 32 via lower side manifold 31.

In such a way, an air-lift pump utilizing transparent tubes 32 and first nozzles 35 causes culture solution C to be circulated between outer vessel 10 and culture solution circulating portion 30.

It is noted that a preferable circulating velocity of culture solution C is such that culture solution C descends within outer vessel 10 at a linear velocity in a range from about 20 cm/s to about 50 cm/s.

Further, heat transfer medium H temperature of which is regulated at a predetermined temperature is supplied from temperature controller 41 into inner vessel 20, performing heat exchange with culture solution C descending within gap 15, with the result that temperature of culture solution C is maintained within a predetermined temperature range. Heat transfer medium H returns to temperature controller 41 after performing heat exchange with culture solution C, being heated or cooled again.

Further to this, a gas to which gas containing carbon dioxide is added is injected from second nozzle 17. Quantity of gas flow from second nozzle 17 is set as to avoid descending of culture solution C within gap 15 from being obstructed.

In addition, a generally uniform sunlight is incident to culture solution C descending within gap 15, after transmitting through the wall of outer vessel 10, from all directions around a vertical direction over 360 degrees while sunlight is also incident to culture solution C ascending within transparent tubes 32, thereby promoting photosynthesis of photosynthesis microorganism and providing an efficient culturing of photosynthesis microorganism. If photosynthesis microorganism is capable of producing a useful substance, a large quantity of useful substance is yielded in cells or other places by photosynthesis.

It is noted that circulation quantity of culture solution C may be reduced by reducing gas feeding quantity from first nozzles 35 and a small quantity of gas, such as air, containing oxygen gas necessary for breathing of photosynthesis microorganism. Although culture solution C requires temperature control little during nighttime due to absence of photosynthesis, it is needless to say that temperature control can be carried out.

Now description on effects of culturing apparatus 100.

According to this embodiment, since culture solution C containing photosynthesis microorganism flows from upside to downside within gap 15 between outer vessel 10 and inner vessel 20 as to form a relatively thin layer, the photosynthesis microorganism in culture solution C can be irradiated efficiently by light such as sunlight from outside of transparent outer vessel 10. This enables photosynthesis microorganism to be cultured to a high concentration, providing an example such that Haematococcus can be cultured, depending on thickness of gap 15, to a high concentration roughly from 5 to 10/L.

In addition, heat exchange with culture solution C in gap 15 performed by heat transfer medium H in inner vessel 20 enables temperature of culture solution C to be maintained at a desired temperature with ease, temperature of culture solution C can be kept at a temperature suitable for culturing without being affected by changes of circumstance caused by, for example, change of season or weather. Therefore, an efficient culturing of photosynthesis, microorganism is achievable.

In particular, the embodiment is advantageous because cooling can be realized efficiently regardless of tendency that temperature of culture solution gets too high in summer, while prior arts are apt to give culture solution a too high temperature over a range suitable for photosynthesis.

Further to this, in the contrast with a tendency that prior arts are apt to give culture solution a too low temperature in winter, the embodiment is advantageous because temperature of culture solution is maintained within a range suitable for photosynthesis in generally the same way as described above. As a result, the culturing apparatus In addition, the embodiment can carry out culturing regardless of summer or wintertime.

Since inner vessel 20 is made of a metal or glass material, which has a higher heat conductivity as compared with that of resin or the like, temperature regulation of culture solution C by temperature controller 41 can be performed extremely quickly and surely.

Gap between outer vessel 10 and inner vessel 20 has a uniform thickness abound a vertical axis, because outer vessel 10 and inner vessel 20 are cylinders coaxially. arranged to each other. This brings a highly uniform current of culture solution, resulting in a desirable situation such that problems such as flow stopping or deposition onto a wall surface scarcely arise.

In addition, since outer vessel 10 and inner vessel 20 extend in a vertical direction and culturing apparatus 100 is a so-called vertical-type apparatus, a large quantity of culture solution can be processed in a small occupation area. Therefore, a plurality of such culturing apparatuses can be arranged in an area, which is not so large, with the result that mass production can be carried out efficiently.

Further, transparent tubes 32 provided by culture solution circulating portion 30 causes culture solution therein to perform photosynthesis by sun light irradiation, enabling a more efficient culturing to be realized.

Still further, first nozzles 35 provided by culture solution circulating portion 30 enables culture solution to be circulated by transparent tubes 32 functioning as an air-lift pump. Such absence of necessity of volute pump or the like can restrain multiplication velocity from being reduced through so-called shear-stress-phenomenon such which is a phenomenon such that multiplication velocity from falls due to mechanical damages of photosynthesis microorganism or external forces such as shear stress applied to photosynthesis microorganism.

In addition, such an air-lift-type pump consumes generally less energy as compared with other pumps.

It is further noted that employment of gas containing carbon dioxide as a gas for air-lifting causes culture solution C to be in contact with bubbles, which contain carbon dioxide and ascend from downside to upside within transparent tubes 32, for a sufficient long time, with the result that culture solution C is supplied with carbon dioxide sufficiently and an enough concentration of dissolved carbon dioxide is maintained as to contribute to photosynthesis of photosynthesis microorganism.

In addition, culture solution C is stirred, thereby preventing deposition of precipitated photosynthesis microorganism from occurring.

Further saying, this embodiment employs outer vessel 10 having a conical (funnel-shaped) bottom portion, which hardly allows photosynthesis microorganism to accumulate.

Still further saying, second nozzle 17 also feeds a gas containing carbon dioxide to culture solution C in outer vessel 10, thereby making not only culturing of photosynthesis microorganism more efficient but also stirring sufficient in generally the same way as the way in case of first nozzles 35.

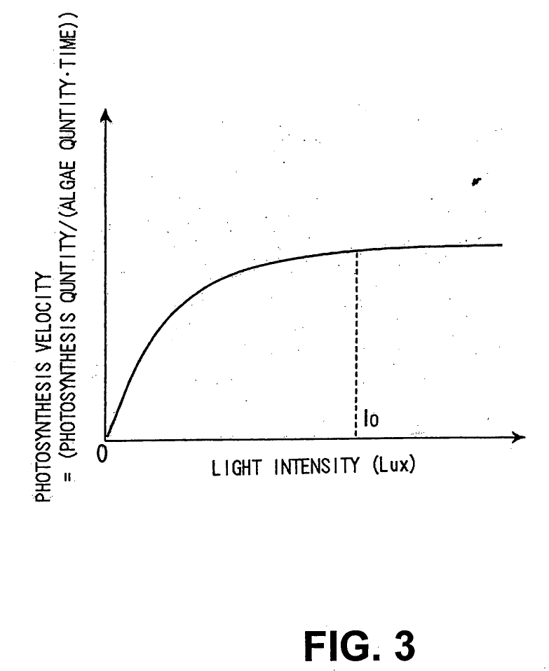

By the way, in general, there is a relation between photosynthesis velocity of photosynthesis microorganism and light intensity as shown in FIG. 3. As understood clearly from FIG. 3, photosynthesis velocity of photosynthesis increases in proportion to light intensity until reaching light saturating point I₀.

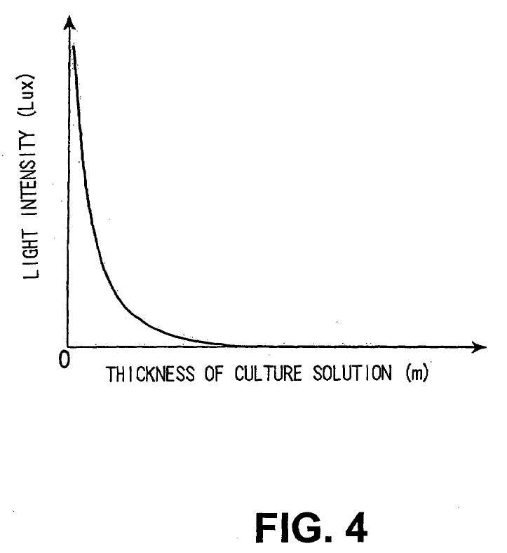

On the other hand, in general, there is a relation between thickness of culture solution and attenuation of light as shown in FIG. 4. As understood clearly from FIG. 4, light is attenuated strikingly with increasing of thickness of culture solution.

Therefore, it is required for making culturing of photosynthesis microorganism efficient that culture solution has a small thickness as to be irradiated by light at a sufficient illuminance.

Thus, according to the embodiment, a thin layer of culture solution C is formed within gap 15, providing a particularly high light utilizing efficiency and a much increased photosynthesis velocity.

It is noted that culturing apparatus 100 of this embodiment employs ventilation opening 38 which can suitably discharges oxygen gas generated by photosynthesis reactions, thereby restrain photosynthesis from being obstructed by excessive dissolved oxygen.

It is noted that the present invention is not limited by this embodiment and allows various modifications.

For example, although the embodiment employs cylindrical outer vessel 10 and cylindrical inner vessel 20, tubes having non-circular cross sections such as rectangular cross may be employed.

Further, although the embodiment employs outer vessel 10 and inner vessel 20 which are arranged coaxially. to each other, vertically extending axes of them may be eccentrically arranged to each other in a manner, for example, such that a wider gap is formed at one side to which sunlight is incident better (for instance, southern side).

Still further, although the embodiment employs outer vessel 10, inner vessel 20 and transparent tubes 32 every axis of which extends in a vertical direction, the present invention can be embodied even if the axis extends in an oblique direction or horizontal direction. If transparent tubes 32 are arranged horizontally, air-lift is not employable, with the result that any other pump has to be employed.

It is also noted that the present invention can be embodied under employment of light-shielding tubes instead of transparent tubes 32 employed in the above-described embodiment in order to have an increased light receiving area.

In addition, allowed are optional changes of the number of transparent tubes 32 depending on concentration of photosynthesis microorganism in culture solution (culture solution concentration) or the like.

Further, a light-shielding cover may be disposed in the vicinity of the bottom portion of outer vessel 10 or lower side manifold 31 may be made of a light-shielding material so that no light is incident to a part of the flowing path of culture solution C, which would bring a cyclic light-and-dark condition under which photosynthesis microorganism is cultured. This can provide an increased culturing efficiency depending on kinds of photosynthesis microorganism.

### EMBODIMENT

### Examples

### Example 1

Isochrysis Galbana, an oceanic microalgae, was cultured outdoors by the use of the above-described culturing apparatus. As for sizes of the culture solution, gap 15 between outer vessel 10 and inner vessel 20 had a thickness of 2.0 cm, a height of 150 cm and each transparent tube 32 had a diameter of 4 cm.

An initial concentration of the algae in the culture solution was 0.5 g/L and 30 L of culture solution falling within a pH range from 7.0 to 8.0 and a temperature range 15° C. to 25° C. was used, and time-mean solar radiation quantity was 14.0 MJ/m² and solar radiation time was 9 hours per 1 day on average, and air to which 1.0 Vol % of carbon dioxide as an additional gas was fed from the nozzles at a rate within a range from 15 L/min to 20 L/min, and culturing period was 14 days.

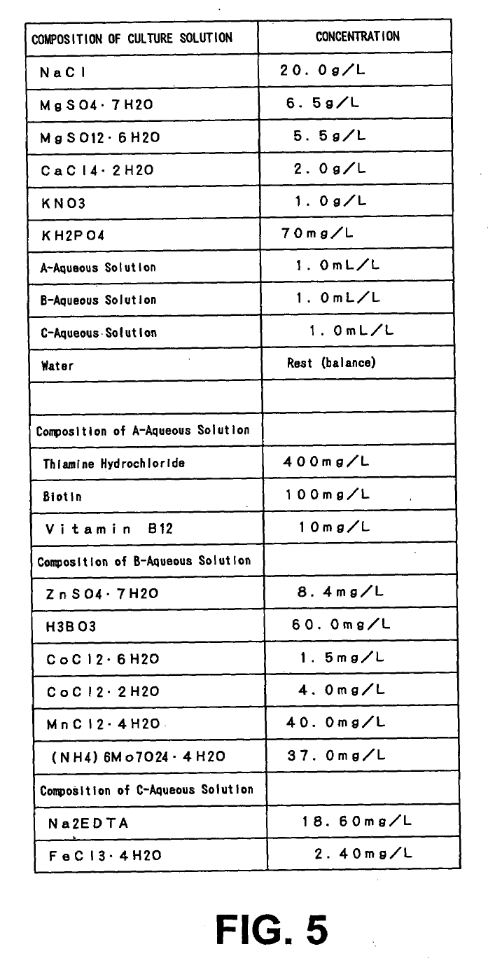

Culture solution shown in FIG. 5 was employed.

The algae had, after being cultured, a concentration reaching a range from 5.0 g/L to 10.0 g/L, having contained DHA (docosahexaenoic acid) in a range from 5 wt % to 8 wt % as a component in evaporated algae.

### Example 2

Spirulina Plantencis, a blue algae, was cultured outdoors by the use of the above-described culturing apparatus. Sizes of the culture solution were the same as those employed in the Embodiment—Example 1.

An initial concentration of the algae in the culture solution was 0.5 g/L and 50 L of culture solution falling within a pH range from 8.5 to 10.5 and a temperature range 25° C. to 35° C. was used, and time-mean solar radiation quantity was 17.0 MJ/m² and solar radiation time was 11 hours per 1 day on average, and air to which 1.0 Vol % of carbon dioxide as an additional gas was fed from the nozzles at a rate within a range from 15 L/min to 25 L/min, and culturing period was 14 days.

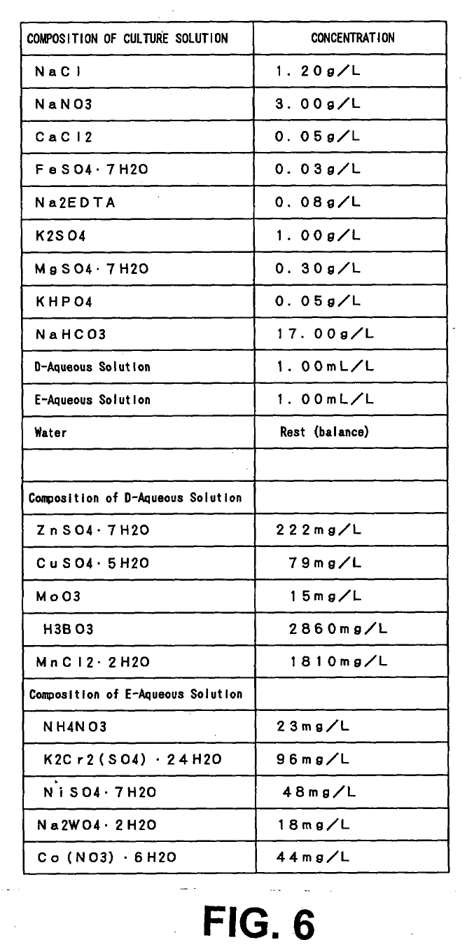

Culture solution shown in FIG. 6 was employed.

The algae had, after being cultured, a concentration reaching a range from 10.0 g/L to 20.0 g/L, having shown a productivity value in a range from 2.0 g/L/day to 5.0 g/L/day.

On the other hand, according to a conventional outdoor-culturing-pond method (ope-pond method), obtained was an algae concentration after being cultured ranging from 0.3 g/L to 0.5 g/L, and a productivity value in a range from 0.1 g/L/day to 0.2 g/L/day.

### Example 3

Haematococcus Pluviaris, a freshwater algae, was cultured outdoors by the use of the above-described culturing apparatus. Sizes of the culture solution were the same as those employed in the Embodiment—Example 1. An initial concentration of the algae (cyst cells) in the culture solution was 0.5 g/L and 50 L of culture solution falling within a pH range from 7.5 to 8.5 and a temperature range 25° C. to 30° C. was used, and time-mean solar radiation quantity was 16.0 MJ/m² and solar radiation time was 12 hours per 1 day on average, and air to which 1.0 Vol % of carbon dioxide as an additional gas was fed from the nozzles at a rate within a range from 25 L/min to 30 L/min, and culturing period was 14 days.

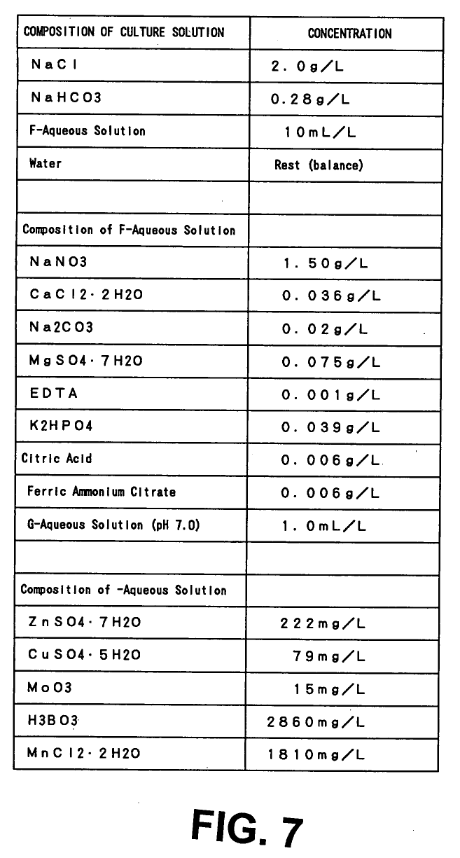

Culture solution shown in FIG. 7 was employed.

The algae had, after being cultured, a concentration reaching a range from 5.0 g/L to 10.0 g/L, having yielded algae (biomass) containing astaxanthin, a carotenoid pigment, in a range from 3 wt % 5 to 8 wt % as a component in evaporated algae.

### Example 4

Nannnochloropsis Oculata, an oceanic microalgae, was cultured outdoors by the use of the above-described culturing apparatus. Sizes of the culture solution were the same as those employed in the Embodiment—Example 1.

An initial concentration of the algae in the culture solution was 0.5 g/L and 50 L of culture solution falling within a pH range from 7.0 to 8.0 and a temperature range 25° C. to 30° C. was used, and time-mean solar radiation quantity was 15.0 MJ/m² and solar radiation time was 11 hours per 1 day on average, and air to which 1.0 Vol % of carbon dioxide as an additional gas was fed from the nozzles at a rate within a range from 25 L/min to 30 L/min, and culturing period was 10 days.

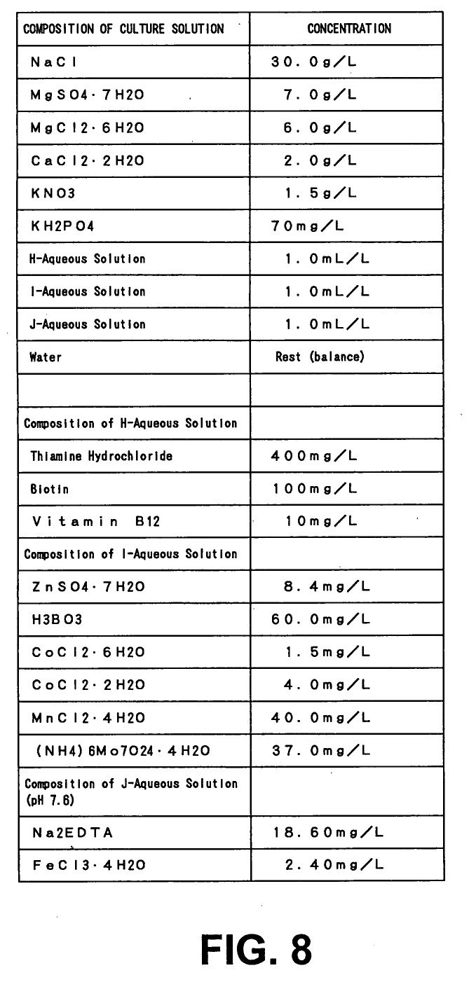

Culture solution shown in FIG. 8 was employed.

The algae had, after being cultured, a concentration reaching a range from 8 g/L to 10 g/L, having yielded algae (biomass) containing polycarboxylic unsaturated fatty acid (EPA) in a range from 5 wt % 8 wt % as a component in evaporated algae. This algae is very useful as feed for multiplying Roatatoria, a botanical plankton, which is used as feed of fry of oceanic cultured fish.

### Example 5

Parietochloris Incisa, which is a freshwater green algae and a kind snow algae, was cultured outdoors by the use of the above-described culturing apparatus. Sizes of the culture solution were the same as those employed in the Embodiment—Example 1.

An initial concentration of the algae in the culture solution was 0.5 g/L and 50 L of culture solution of pH 7.5 and a temperature of 25° C. was used, and time-mean solar radiation quantity was 15.0 MJ/m² and solar radiation time was 11 hours per 1 day on average, and air to which 2.0 Vol % of carbon dioxide as an additional gas was fed from the nozzles at a rate within a range from 25 L/min to 30 L/min, and culturing period was 14 days.

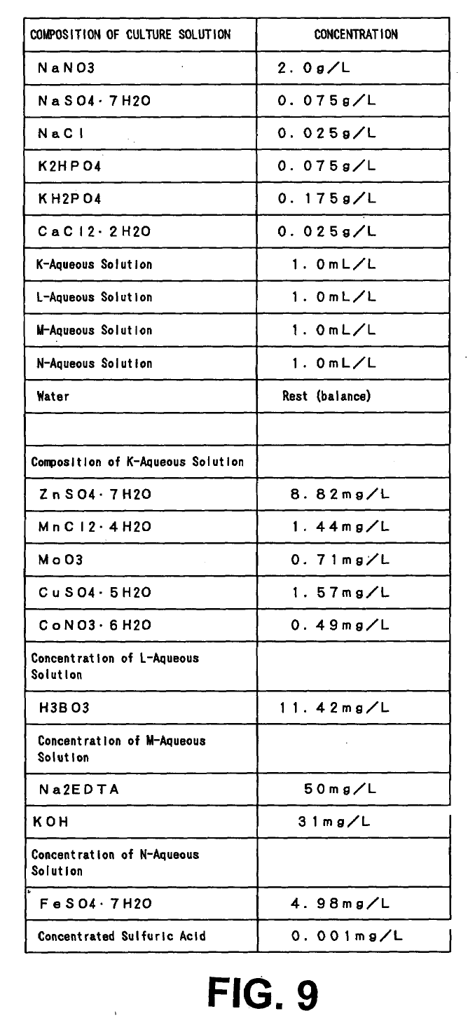

Culture solution shown in FIG. 9 was employed.

The algae had, after being cultured, a concentration reaching a range from 5 g/L to 8 g/L, having yielded algae containing 6.5 wt % of arachidonic acid (ARA) ester contained, which is a kind of polycarboxylic unsaturated fatty acid, as a component in evaporated algae.

### Example 6

Nostoc Commune belonging to Nostoc genus was cultured outdoors by the use of the above-described culturing apparatus. Sizes of the culture solution were the same as those employed in the Embodiment—Example 1.

An initial concentration of the algae in the culture solution was 0.5 g/L and 50 L of culture solution falling within a pH-range from 7.5 to 8.0 and a temperature of 25° C. was used, and time-mean solar radiation quantity was in a range from 7 MJ/m² to 10 MJ/m² and solar radiation time was 9 hours per 1 day on average, and air to which 1.0 Vol % of carbon dioxide as an additional gas was fed from the nozzles at a rate within a range from 25 L/min to 30 L/min, and culturing period was 14 days.

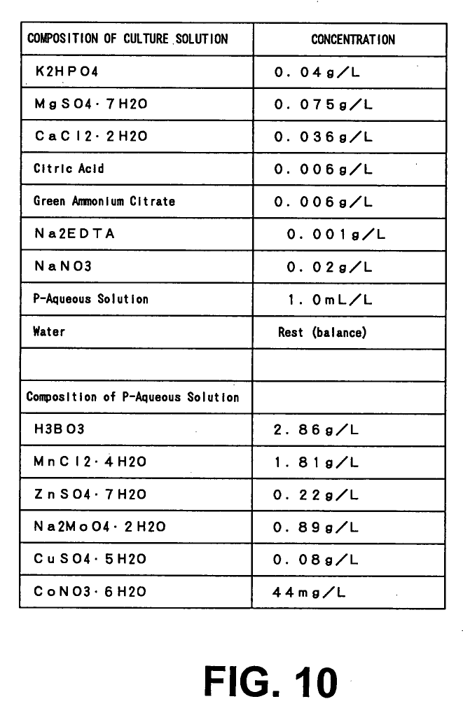

Culture solution shown in FIG. 10 was employed.

The algae had, after being cultured, a concentration reaching a range from 4 g/L to 5 g/L, having yielded algae containing polysaccharide in a range from 10 wt % to 15 wt % as a component in evaporated algae. Polysaccharide was hot-water-extracted. According to an analysis, polysaccharide contained plenty of β-1,3-glucan concentration of which fell within a range from 3 wt % to 4 wt % as a component in evaporated algae.

### Example 7

Phaeodactylum tricornutum, an oceanic algae, was cultured outdoors by the use of the above-described culturing apparatus. Sizes of the culture solution were the same as those employed in the Embodiment—Example 1.

An initial concentration of the algae in the culture solution was 0.3 g/L and 50 L of culture solution falling within a pH-range from 7.5 to 8.5 and a temperature of 26° C. was used, and time-mean solar radiation quantity was 15.0 MJ/m² and solar radiation time was 11 hours per 1 day on average, and air to which 1.0 Vol % of carbon dioxide as an additional gas was fed from the nozzles at a rate within a range from 25 L/min to 30 L/min, and culturing period was 14 days.

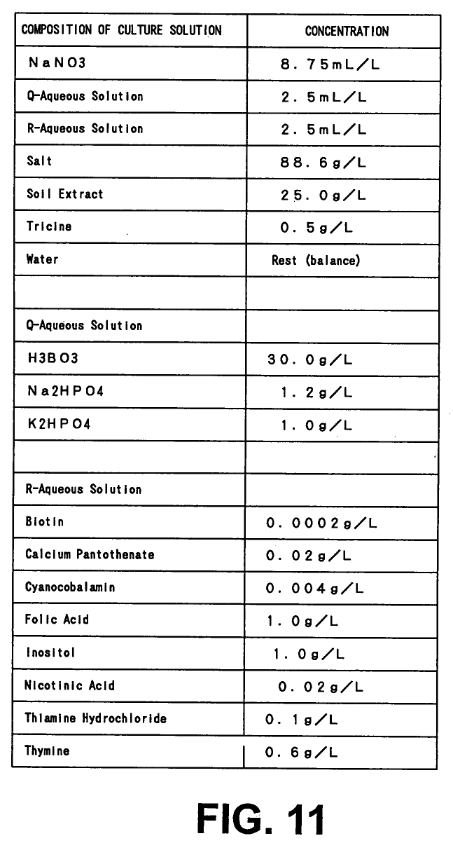

Culture solution shown in FIG. 11 was employed.

The algae had, after being cultured, a concentration reaching a range from 5 g/L to 7 g/L.

Phaeodactylum is a very useful oceanic microalgae, as well as Cheatoceros gracilis belonging to Cheatoceros genus, as feed of Bivalvia and Crustacea such as abalone, lobster.

### INDUSTRIAL UTILITY

The present invention provides a culturing apparatus and a culturing method capable of culturing photosynthesis microorganisms from which various useful components, for example, carotenoids such as β-carotene, or pigments such as astaxanthin, or polycarboxylic unsaturated fatty acids such as EPA, DHA or ARA, or polysaccharides such as β-1,3-glucan, can be extracted. Further, such photosynthesis microorganisms are useful in themselves for various usages such as feed of fry of fishes or shellfishes.

1. A culturing apparatus for photosynthesis microorganism comprising:
a transparent outer vessel which accommodates culture solution containing photosynthesis microorganism and is configured like a cylinder extending in a predetermined axis-direction;
an inner vessel which is disposed within said outer vessel and configured like a cylinder extending in said axis-direction:
a culture solution circulating portion which draws said culture solution from one side along said axis-direction in the outer vessel and supplies the drawn culture solution to the other side along said axis-direction in the outer vessel; and
a heat transfer medium feeding portion which supplies heat transfer medium into said inner vessel.

2. A culturing apparatus for photosynthesis microorganism according to claim 1, wherein said outer vessel and said inner vessel are arranged coaxially.

3. A culturing apparatus for photosynthesis microorganism according to claim 1, wherein both said outer vessel and said inner vessel are cylinders.

4. A culturing apparatus for photosynthesis microorganism according to claim 1, wherein said inner vessel is made of a metal material or a glass material.

5. A culturing apparatus for photosynthesis microorganism according to claim 1, wherein said inner vessel is made of a resin material.

6. A culturing apparatus for photosynthesis microorganism according to claim 1, wherein said culture solution circulating portion is provided with a transparent and cylindrical tube through which said culture solution flows.

7. A culturing apparatus for photosynthesis microorganism according to claim 1, wherein said axis-direction is a vertical direction.

8. A culturing apparatus for photosynthesis microorganism according to claim 1, wherein said culture solution circulating portion is provided with a transparent tube, in which said culture solution flows, and a first nozzle,
said transparent tube being configured like a cylinder extending in a vertical direction, and
said first nozzle being disposed at a lower part of said transparent tube and supplying a gas into said transparent tube.

9. A culturing apparatus for photosynthesis microorganism according to claim 7, wherein a second nozzle supplying a gas is disposed at a bottom part of said outer vessel.

10. A culturing apparatus for photosynthesis microorganism according to claim 1, wherein said heat transfer medium is heating medium or cooling medium.

11. A method of culturing photosynthesis microorganism, wherein said photosynthesis microorganism is cultured by the use of a culturing apparatus according to claim 1.
